(19) United States
(12) Patent Application Publication
(10) Pub. No.: US 2010/0274175 A1
(43) Pub. Date: Oct. 28, 2010

(54) METHOD AND APPARATUS FOR SKIN ABSORPTION ENHANCEMENT AND TRANSDERMAL DRUG DELIVERY

(75) Inventor: Gian Franco Bernabei, Florence (IT)

Correspondence Address: FOLEY AND LARDNER LLP;SUITE 500, 3000 K STREET NW, WASHINGTON, DC, 20007, US

(73) Assignee: MATTIOLI ENGINEERING LTD.

(21) Appl. No.: 12/762,196

(22) Filed: Apr. 16, 2010

Related U.S. Application Data
(60) Provisional application No. 61/171,539, filed on Apr. 22, 2009.

(51) Int. Cl. A61N 1/30

(52) U.S. Cl. 604/20

(57) ABSTRACT

A method and apparatus for enhancing absorption of a substance to be provided on a region of a patient's skin, includes outputting, by an electronic burst pulse generator, bursts of electronic pulses to the patient's skin, wherein the electronic pulses are generated by an electronic pulse generator. The method and apparatus also includes outputting, by a mechanical burst pulse generator, bursts of mechanical vibrations to the patient's skin at the same time the electronic pulses are applied to the patient's skin. The bursts of electronic pulses are output at a first burst rate, and the mechanical vibrations are output as bursts of vibrations at a second burst rate.

### CROSS-REFERENCE TO RELATED APPLICATION

This application claims the benefit under 35 U.S.C. §119(e) to U.S. Provisional Application No. 61/171,539, filed on Apr. 22, 2009, which is incorporated by reference herein in its entirety.

### BACKGROUND OF THE INVENTION

1. Field of the Invention

The invention relates to application of a substance to a patient's skin, whereby the substance is an ascorbic acid, lidocaine, collagen, or other type of skin treatment substance.

2. Description of the Related Art

It is known that an electrical pulse applied to the skin is useful in order to increase the absorption of a substance previously applied to the skin, whereby this technique is known as electroporation. Such a substance to be applied to the skin may be a liquid, a gel, a lotion, or a cream, for example.

It is also known that a mechanical pulse can be applied to the skin at the same time as the electrical pulse, in order to increase the effectiveness of skin absorption.

It is desired to provide an apparatus and a method to increase the absorption of a substance to be applied to the skin, in order to obtain an increased (e.g., moisturizing) affect of the substance applied to the skin, as well as to obtain a fairly even absorption of the substance to the skin.

### SUMMARY OF THE INVENTION

The present invention is directed to an apparatus and a method for enhancing the absorption of a substance to be applied on the skin.

According to one aspect of the invention, there is provided an apparatus for enhancing absorption of a substance to be provided on a region of a patient's skin. The apparatus includes a head portion having at least one electrode that outputs bursts of electronic pulses to the patient's skin, whereby the electronic pulses are generated by an electronic pulse generator. The apparatus also includes a mechanical vibrator that generates mechanical vibrations to be applied to the patient's skin at the same time the electronic pulses are applied to the patient's skin. The bursts of electronic pulses are output at a first burst rate, and the mechanical vibrations are output as bursts of vibrations at a second burst rate.

According to another aspect of the invention, there is provided a method of transdermal drug delivery to be provided to a patient's skin. The method includes applying at least one burst of electronic pulses to the patient's skin at a first burst rate. The method also includes applying at least one burst of mechanical vibrations to the patient's skin at a second burst rate, in which the mechanical vibrations are applied to the patient's skin at the same time the electronic pulses are applied to the patient's skin.

### BRIEF DESCRIPTION OF THE DRAWINGS

The foregoing advantages and features of the invention will become apparent upon reference to the following detailed description and the accompanying drawings, of which:

FIG. 1 shows such a hand-held probe that can be utilized to provide electrical pulses to a patient's skin, in accordance with an embodiment of the present invention;

FIG. 2 shows a first burst of mechanical vibrations and a second burst of mechanical vibrations that are output to a patient's skin, in accordance with an embodiment of the present invention;

FIG. 3 shows such a mechanical burst pulse vibration frequency spectrum, in accordance with an embodiment of the present invention;

FIG. 4 shows an electrical diagram of a pulse generator that provides electrical pulses to an array of electrodes disposed on a vibrating plate provided at a head-end of the probe, in accordance with an embodiment of the present invention;

FIG. 5A shows a side view of a mechanical vibration unit in accordance with an embodiment of the present invention;

FIG. 5B shows a front view of the mechanical vibration unit in accordance with an embodiment of the present invention; and

FIG. 6 is a block diagram of a skin treatment apparatus in accordance with an embodiment of the present invention.

### DETAILED DESCRIPTION OF THE PREFERRED EMBODIMENTS

Preferred embodiments of the invention will be described in detail below, with reference to the accompanying drawings.

U.S. Pat. No. 7,376,460, which is incorporated in its entirety herein by reference, describes a method and an apparatus for skin treatment of a patient. In the '460 patent, mechanical vibrations are continuously applied to a patient's skin, while electronic bursts of pulses are applied to the patient's skin at the same time. The mechanical vibrations are applied at a vibration rate that is an integer multiple or submultiple with respect to the burst rate of the electronic pulses applied to the patient's skin.

Experimental results performed by the inventor have determined that applying the mechanical vibrations to a patient's skin in pulse bursts at a mechanical vibration burst rate, at the same time the electrical pulses are applied to the patient's skin, enhances the skin absorption effect of a drug that is provided to the skin while these mechanical and electrical vibrations are also being applied to the skin.

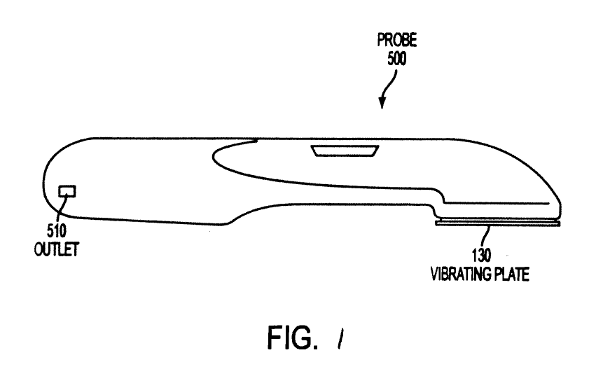

The electrical pulses are preferably applied to the patient's skin by an array of electrodes that are provided on a head of a skin treatment device, such as a hand-held probe. FIG. 1 shows such a hand-held probe 500 that can be utilized to provide electrical pulses to a patient's skin in accordance with the present invention. The hand-held probe 500 includes an outlet 510 for connecting to an electrical outlet, and a vibrating plate 130 provided on a head of the probe 500. The probe 500 can be battery-powered, so that connection to an electrical outlet is not required. The vibrating plate contains a piezoelectric material that is driven by an additional pulse generator at a frequency of 33 KHz. The combination of an eccentric motor and the piezoelectric material generate a vibration spectrum composed of two carrier at 100 Hz and 33 KHz, where the carrier at 33 KHz is modulated by a 100 Hz square wave. Other frequency values other than 33 KHz may be utilized for driving the piezoelectric material, such as 25 KHz, 50 KHz, 66 KHz, etc., while remaining within the spirit and scope of the invention.

As described in U.S. Pat. No. 7,376,460, the use of a continuous stream of mechanical vibrations at the same time that the electrical pulses are applied to skin, and at a same or nearly the same frequency as the burst pulse rate, results in a patient having a greater tolerance to the strength (current and voltage) of the electrical pulses applied to the patient's skin. For example, using a electrical pulse burst rate of 50 Hz (that is the rate between bursts of pulses), mechanical vibrations may be provided at a range of between 40 to 60 Hz at the same time that the electrical pulse bursts are applied to the skin, to provide a “masking effect.” U.S. Pat. No. 7,376,460 describes that utilizing mechanical vibrations at or around (e.g., +/−10% of) the fundamental frequency of the electrical pulse burst rate, at or around the first harmonic of the electrical pulse burst rate, at or around the second harmonic of the electrical pulse burst rate, and/or at or around the third harmonic of the electrical pulse burst rate, gives the patient a “good sensation” so that he/she can tolerate a higher strength of electrical pulses being applied to his/her skin at the same time. Thus, for a 50 Hz electrical pulse burst rate, mechanical vibrations may be applied to the patient's skin at the same time, with the mechanical vibration rate being either 40 to 60 Hz, 90 to 110 Hz, 140 to 160 Hz, and/or 190 to 210 Hz. By having mechanical vibrations applied to the patient's skin at the same time that the electrical pulse bursts are applied to patient's skin, the patient's discomfort level caused by the tinging sensation of the electrical pulses is lessened (e.g., masked somewhat).

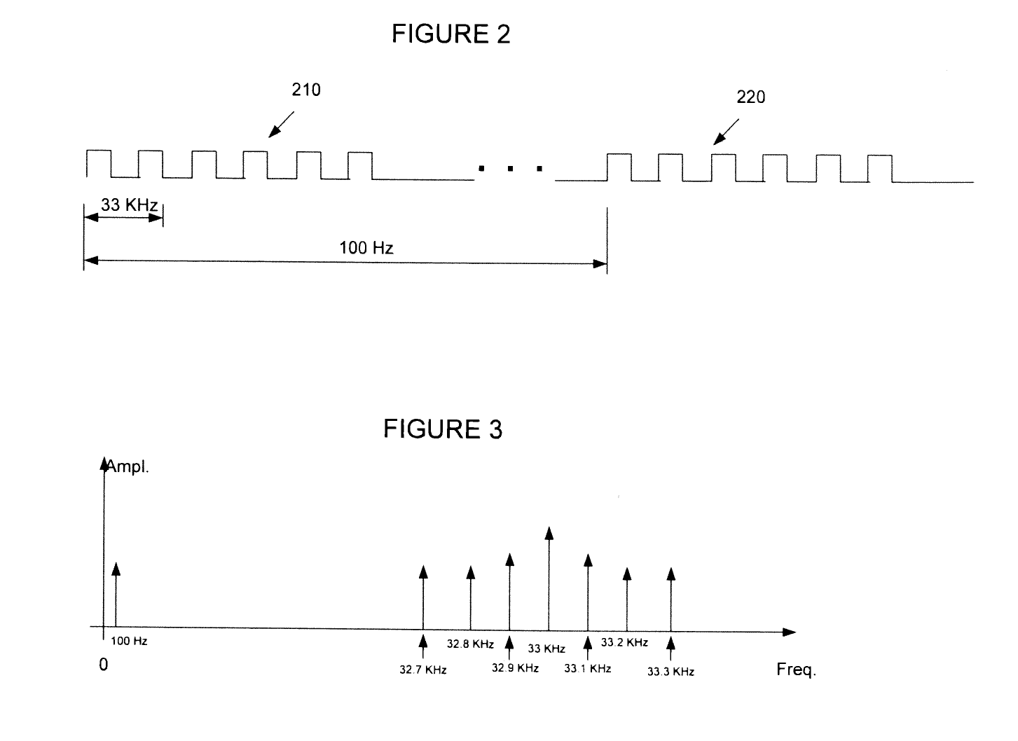

In a first embodiment of the invention, the mechanical vibrations are provided in bursts of mechanical vibrations, as opposed to a continuous stream of mechanical vibrations. FIG. 2 shows a first burst of mechanical vibrations 210 and a second burst of mechanical vibrations 220 that are output to a patient's skin, whereby the burst rate is 100 Hz (that is, 100 mechanical bursts per second). The mechanical vibration pulses provided in each burst of mechanical vibrations are provided at a rate of 33 KHz (that is, 33,000 mechanical vibrations per second). The much higher mechanical vibration burst rate, as compared to the continuous stream of mechanical vibrations described in U.S. Pat. No. 7,376,460, provides for increased heat to the fatty tissue in the patient's skin, and thereby provides for a better skin absorption effect for a drug applied to the patient's skin, and it also provides for a better fatty tissue breakdown in the patient's skin.

The mechanical vibrations in each burst of mechanical vibrations need not be at an integer multiple or submultiple with respect to the bursts of electronic pulses applied to the patient's skin at the same time. For a 100 Hz mechanical pulse burst rate having 33 KHz mechanical vibration pulses in each of the bursts, one obtains a frequency spectrum of a 33 KHz carrier with harmonics of 100 Hz. That is, the mechanical vibration frequency spectrum for this example corresponds to a center frequency at 33 KHz, a first harmonic at 33.10 KHz and 32.90 KHz, a second harmonic at 33.200 KHz and at 32.800 KHz, a third harmonic at 33.30 KHz and 32.70 KHz, etc. FIG. 3 shows such a mechanical vibration frequency spectrum 200.

The effect of applying the probe to the skin is that the skin vibrates due to the electrical pulses applied by way of the array of electrodes (for example, the array of electrodes shown in FIGS. 2A and 2B of U.S. Pat. No. 7,376,460), and also due to the mechanical pulses applied to the patient's skin at the same time. The electrical pulses are preferably applied at a fixed frequency between 200 and 10,000 Hz (optimally at a frequency value between 2,000 to 3,000 Hz), and are grouped in burst of pulses (e.g., each burst may correspond to 100 to 1000 separate pulses that have opposite polarities with respect to adjacent pulses in the same burst of pulses). The ON time of each burst is a fixed value between 5 to 50 milliseconds, and the OFF time between two consecutive bursts is a fixed value between 5 to 50 milliseconds (the burst ON time can be 10 milliseconds and the OFF time between consecutive bursts can be 10 milliseconds).

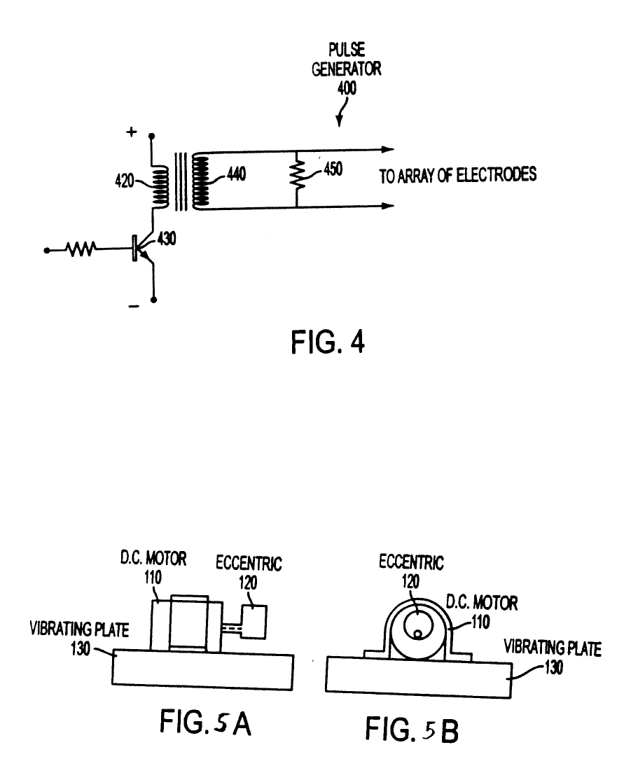

As described above, the electrical pulses applied to the skin by way of the electrodes can be exponential pulses with peak-to-peak voltage of 160 V at a fixed frequency between 2,000 to 3,000 Hz. One way of providing such electrical pulses is by an electrical structure that corresponds to a pulse generator 400 as shown in FIG. 4, in which a transformer is used as an element of a pulse generator. The transformer, as well as the other elements of the pulse generator, can be housed within the hand-held probe.

Along with the electrical pulses applied to the skin, a mechanical vibration is also provided to the skin in the first embodiment in order to increase the absorption of a substance that is applied on the skin.

The absorption effect is enhanced by the simultaneous increase of transpiration, whereby the absorption effect is greatest when the mechanical vibration is synchronized in phase and in frequency with the electric pulse application. Thus, in the example discussed above, while the electrical burst of pulses (at 2,200 Hz) are provided to the skin at a burst ON/OFF frequency, e.g., 50 or 100 Hz, by way of an electrode array, the skin is also mechanically vibrated at the same frequency, e.g., 50 or 100 Hz, by way of the vibrating plate, but whereby the mechanical vibrations are output as bursts of pulses at a high vibration rate, such as 33 KHz. The mechanical burst vibration and the electrical burst application can be provided in phase with respect to each other, in order to increase the skin absorption effect.

Thus, in the example discussed above, while the burst of electrical pulses are provided to the skin by way of the electrode array, the skin is also mechanically vibrated at the same frequency by way of the vibrating plate that provides bursts of mechanical vibrations to the patient's skin. The mechanical vibration and electrical pulse application can be provided in phase with respect to each other, in order to increase the skin absorption effect.

Moreover, the absorption effect is further enhanced when the mechanical vibration is applied orthogonal to the surface of the skin. While Applicant does not intend to be tied down to any particular theory of operation, one possible explanation of the physical phenomena of one or more embodiments of the present invention is that, while the electrical pulses “stretch” the skin, thus increasing periodically the diameter of the pores of the skin, at the same time the mechanical vibration “pumps” the substances (gel, liquid or cream) inside the skin (through the opened pores). The mechanical and electrical synchronization achieves the effect that the “pumping” action (due to the mechanical stimulation of the skin) takes place at the same instant in time that the pores are at their maximum “open” diameter (due to the electrical stimulation of the skin).

The apparatus according to a first embodiment the present invention includes a probe having two main parts:

A) a handle containing a power source (e.g., batteries) and a pulse generator; and

B) a vibrating head containing components for generating the bursts of mechanical vibrations and also containing an array of electrodes (see FIG. 3 of U.S. Pat. No. 7,376,460, for example, which shows one possible probe head structure for providing mechanical vibrations and electrical pulses to a patient's skin, whereby these are provided in bursts of vibrations and pulses in the first embodiment of the present invention).

The vibrating head, in a preferred configuration of the first embodiment, includes a D.C. electrical motor for generating vibrations to the skin, and a piezoelectric transducer. FIGS. 5A and 5B respective show a side view and a front view of a D.C. electrical motor 110, in which a rotating shaft of the D.C. electrical motor 110 is an eccentric 120 to thereby provide eccentric motion. The eccentric motion, during rotation of the D.C. electrical motor 110, generates a vibration onto the vibrating plate 130 (that is directly coupled to the D.C. electrical motor 110) that is at the same frequency of the rotation of the D.C. electrical motor 110 (e.g., 50 Hz or 60 Hz or some other desired frequency). Other ways of causing vibrations in synchronization with the providing of electrical pulses to a patient may be contemplated while remaining within the scope of the invention. Note that the use of mechanical pulses as bursts of pulses at the same or nearly the same rate as bursts of electrical pulses, but not necessarily in synchronism with each other, as described earlier, provides a good effect in that it lessens the patient's discomfort level associated with the buzzing and tinging sensation caused by receiving electrical pulses to the skin alone. Also, the use of adjacent pulses in each burst of opposite polarity to each other results in no current buildup to the patient's skin, which can be a detrimental effect of conventional devices that use electrical pulses of the same polarity to be provided to a patient's skin. The D.C. electrical motor 110 provides a continuous stream of vibrations, such as a stream of 33 KHz vibrations, whereby a piezoelectric transducer (not shown in FIGS. 5A and 5B) receives the stream of electrical pulses and outputs burst of mechanical vibrations at a prescribed burst rate, such as at a 50 Hz or 100 Hz burst rate.

FIG. 4 shows circuitry for providing electrical pulses to an array of electrodes provided on a head of a probe. The circuitry of FIG. 4 corresponds to a pulse generator 400, and is preferably disposed within the housing of the probe 500 of FIG. 1. The electrical pulses generated by the pulse generator 400, when those pulses are provided to the skin, can be flat-shaped or exponentially-shaped pulses with peak-to-peak current of 20 mA and with peak-to-peak voltage of 160 V at a frequency of between 2,500 Hz to 3,000 Hz. Of course, other peak-to-peak currents (e.g., 5 mA to 40 mA) and operating frequencies (500 Hz to 15,000 Hz) may be employed, while remaining within the spirit and scope of the invention as described herein. Alternatively, square waves, sawtooth or sinusoidal pulses can be provided to the electrodes.

FIGS. 5A and 5B show the vibrating plate 130 that is physically coupled to the D.C. electrical motor 110. The vibrating plate 130 can be 50×50 mm in size (other sizes are possible while remaining within the scope of the invention), where parallel metallic stripes are deposited on it, in order form the array of electrodes. The vibrating plate 130 is caused to vibrate at the same phase and frequency as the electrical pulses provided to the skin by way of the array of electrodes (disposed on the vibrating plate), in order to enhance the skin absorption effect.

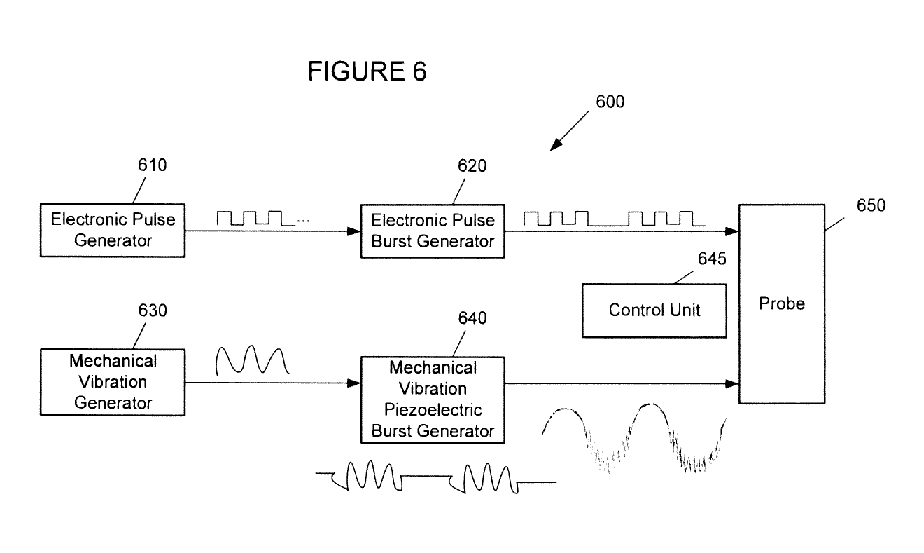

FIG. 6 is a block diagram of a skin treatment apparatus according to the first embodiment. An electronic pulse generator 610 provides a continuous stream of electronic pulses (see FIG. 4, for example), and an electronic pulse burst generator 620 generates bursts of electronic pulses at a prescribed first burst rate (e.g., 50 Hz, 100 Hz). A mechanical vibration device 630 provides a continuous stream of mechanical vibrations (see FIGS. 5A and 5B, for example), and a mechanical vibration piezoelectric burst generator 640 generates bursts of mechanical pulses at a prescribed second burst rate (e.g., 50 Hz, 100 Hz). The electronic pulses and the mechanical pulses in each of the respective electronic pulse bursts and mechanical pulse bursts are at a much higher rate (e.g., in the KHz range) than the respective burst rates (which are in the 10 s of Hz for practical applications). A control unit 645 sets the electronic and mechanical pulse rates, as well as the electronic and mechanical burst pulse rates, by way of control signals provided to the devices 610, 620, 630 and 640. A probe 650 receives the mechanical and electronic pulse bursts and provides them to a patient's skin at the same time via an array of electrodes and a vibrating head of the probe 650.

Different embodiments of the present invention have been described according to the present invention. Many modifications and variations may be made to the techniques and structures described and illustrated herein without departing from the spirit and scope of the invention. Accordingly, it should be understood that the apparatuses described herein are illustrative only and are not limiting upon the scope of the invention. For example, the frequency of the mechanical vibration and the frequency of the bursts of electronic pulses may be the same, as described above with respect to several different embodiments, or they may be an integer multiple or submultiple of each other. For example, an electronic pulse burst frequency of 50 Hz may be utilized together with a mechanical vibration burst frequency of 100 Hz (each having mechanical and electrical pulses provided in each burst at a rate of between 20 KHz to 50 KHz, for example), and still one would achieve an effect of increased absorption and decrease in skin sensitivity (e.g., lowering of the pain) to the patient. Alternatively, an electronic burst frequency of 200 Hz may be utilized together with a mechanical vibration burst frequency of 100 Hz, and still one would achieve an effect of increased absorption and decrease in skin sensitivity. Also, the plate on which the electrodes are disposed on the probe in certain embodiments may be a sterilized disposable part (e.g., removed from a sterilized container and then affixed to the head of the probe). In this implementation, when one is finished treating a patient, the disposable plate is removed from the probe and discarded, and then a new sterilized plate is affixed to the probe (with the electrodes provided thereon) in order to treat another patient. By such an implementation, this greatly reduces the possibility of contamination between different patients, since the portion of the probe directly in contact with each patient is discarded after treatment of each patient.

What is claimed is:

1. An apparatus for enhancing absorption of a substance to be provided on a region of a patient's skin, comprising:
a head portion having at least one electrode that outputs bursts of electronic pulses to the patient's skin, wherein the electronic pulses are generated by an electronic pulse generator;
a mechanical vibrator that generates mechanical vibrations to be applied to the patient's skin at the same time the electronic pulses are applied to the patient's skin,
wherein the bursts of electronic pulses are output at a first burst rate, and the mechanical vibrations are output as bursts of mechanical vibrations at a second burst rate.

2. The system according to claim 1,
wherein the electronic pulse generator is a transformer that provides for pulses to be output in alternate polarities in each of the sequence of bursts, and
wherein no pulses are output by the electronic pulse generator in between consecutive bursts of electrical pulses.

3. The system according to claim 1, further comprising:
a mechanical pulse burst generator configured to deliver a spectrum of vibrations between 50 Hz and 50 KHz.

4. The system according to claim 3, further comprising:
a vibrating plate configured to receive the bursts of mechanical vibrations output by the mechanical pulse burst unit, and to provide the bursts of mechanical vibrations to the patient's skin.

5. The system according to claim 1, wherein no pulses are output by the mechanical vibrator in between consecutive bursts of mechanical pulses.

6. The system according to claim 1, wherein the first burst rate corresponds to a value between 2000 and 3000 Hz, and wherein the second burst rate corresponds to either 50 Hz or 100 Hz.

7. The system according to claim 1, wherein the mechanical vibrator is a piezoelectric vibrator.

8. A method for enhancing absorption of a substance to be provided on a region of a patient's skin, comprising:
outputting bursts of electronic pulses to the patient's skin, wherein the electronic pulses are generated by an electronic pulse generator;
outputting bursts of mechanical vibrations to the patient's skin at the same time the electronic pulses are applied to the patient's skin,
wherein the bursts of electronic pulses are output at a first burst rate, and the mechanical vibrations are output as bursts of vibrations at a second burst rate.

9. The method according to claim 8,
wherein electronic pulses in each of the bursts of electronic pulses are output in alternate polarities, and
wherein no pulses are output by the electronic pulse generator in between consecutive bursts of electrical pulses.

10. The method according to claim 8, wherein no pulses are output in between consecutive bursts of mechanical pulses.

11. The method according to claim 8, wherein the first burst rate corresponds to a value between 2000 and 3000 Hz, and wherein the second burst rate corresponds to either 50 Hz or 100 Hz.

12. The method according to claim 8, further comprising:
creating bursts of mechanical vibrations to be then outputted to the patient's skin.

13. The method according to claim 12, wherein the burst of mechanical vibrations are created by supplying a continuous stream of mechanical vibrations to a piezoelectric burst generator.
